(19) United States
(12) Patent Application Publication
(10) Pub. No.: US 2006/0029641 A1
(43) Pub. Date: Feb. 9, 2006

(54) Calcium and magnesium nutritional supplement

(76) Inventors: Nathan I. Keller, Orem, UT (US); W. Ivan Keller, Orem, UT (US); Jonathan S. Keller, Orem, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 10/912,425

(22) Filed: Aug. 5, 2004

(51) Int. Cl. A61K 47/00; A61K 33/06

(52) U.S. Cl. 424/439; 424/682

(57) ABSTRACT

The present invention provides a method and composition for dietary mineral supplementation utilizing a form and dosage of calcium and magnesium for enhanced calcium absorption. The method includes the steps of delaying the absorption of the magnesium for a period of time after absorption of the calcium component of the supplement. The supplement provided herein facilitates absorption of calcium in the teeth and bones of a subject, for enhanced physiological and psychological benefits. The supplement is especially beneficial for subjects experiencing osteoporosis, arthritis, demineralization of teeth and bones, bodily pain and lack of energy, as well as for the prevention of these ailments.

### BACKGROUND OF THE INVENTION

1. Field of the Invention

The present invention relates generally to an oral nutritional supplement containing calcium and magnesium, and more particularly, to a nutritional supplement in which the absorption of one of the calcium or magnesium is delayed within the digestive track in order to allow absorption of one of the calcium or magnesium prior to absorption of the other.

2. Description of the Related Art

Minerals, which are primarily stored in bone and muscle tissue, must be replenished daily. Replenishing minerals appropriately can be a complex task and research has shown that there must be a balance of minerals taken daily for optimal absorption. One key factor in determining a nutritional supplement composition relates to the interaction of different vitamins and minerals. For example, a large dose of one “B” vitamin may cause a deficiency in another “B” vitamin. The same is true for minerals, which must also be taken in correct amounts and combinations to produce the synergistic effect of increasing the positive effect of each one alone.

Amongst vitamins and minerals, calcium is one of the most important minerals in a balanced diet and is vital to the formation of bones and teeth. Calcium is also important for numerous other physiological functions such as the regulation of heartbeat, transmission of nerve impulses, lowering cholesterol, prevention of cardiovascular disease, muscular growth, muscle repair and contractions, maintenance of energy, protein structuring of RNA and DNA, proper cell permeability, the health of skin, hair, nails and gums, improvement in blood clotting, prevention of osteoporosis and cancer, neuromuscular activity, reduction of high blood pressure, breakdown of fats, revitalization of the body from the negative effects of alcohol and tobacco, and the prevention of lead absorption in bones and teeth. For psychological functions, calcium can help prevent nervousness, depression, hyperactivity and can even help induce sleep at night. Calcium deficiency can lead to osteoporosis, joint pain, rheumatoid arthritis, muscle cramps, heart palpitations, tooth decay, hypertension, mental dullness and mental disorders of many types. One reason for calcium deficiency is that throughout our lives, bone is constantly being broken down and repaired. Therefore, with the formation of new bone, a constant intake of calcium is vital.

In the United States and other modern countries, nearly 80-85% of all adults are believed to be deficient in calcium intake by diet alone. The average American is estimated to be at least 25% deficient in calcium. It is reported that up to 50% of women ages 45-75 have some form of osteoporosis. For children of ages 9-18 years and adults over 50, calcium is especially needed in high concentrations. Research shows that children ages 9-18 need at least 1300 mg of calcium per day, adults ages 19-50 need at least 1000 mg per day, and adults over 50 need at least 1200 mg of calcium per day.

Even if a recommended dose of calcium is ingested each day, absorption of the calcium can be inhibited by the intake of soft drinks, alcohol, tobacco, various “junk foods,” processed foods, overcooked foods, smoking, caffeine in foods and canned foods. Moreover, medication, chemotherapy, lack of exercise, diets that are high in fat, sugars, excess salt, white flour and certain diseases can also inhibit the absorption of calcium. Furthermore, as studies have indicated, the simultaneous ingestion of calcium and magnesium in combination results in competitive absorption of the two minerals in which neither is fully absorbed. If the body does not receive calcium, it simply takes the calcium needed from bones and teeth, which store nearly 99% of the calcium in our body.

It is apparent that there are many calcium supplements on the market which aim to increase a subject's daily intake of calcium. More recently, however, it has been recognized that magnesium is needed for calcium absorption. Dr. Nan Kathryn Fuchs wrote, “Without enough magnesium calcium can collect in the soft tissues and cause one type of arthritis. Not only does calcium collect in the soft tissues of arthritics, it is poorly, if at all, absorbed into their blood and bones. But taking more calcium is not the answer; it only amplifies the problem. In fact, excessive calcium intake and insufficient magnesium can contribute to both of these diseases.”

It has also been recognized that calcium and magnesium may actually compete for absorption. Thus, it has been suggested in the art to take magnesium and calcium supplements at different times to allow for better absorption of each of these minerals. Such staggered dosing, however, is often quite inconvenient and not very practical. Moreover, such teachings lack specific guidelines for outlining how far apart in time the two minerals should be taken.

Many common calcium supplements that are in pill form also contain magnesium, which as previously discussed has been shown to be an important mineral for bone density and strength. Thus, despite the fact that a particular supplement contains both calcium and magnesium in recommended dosages, the supplement itself may interfere with the absorption of calcium which could lead to further calcium deficiency.

In most supplement formulas, 10-40% of calcium is elemental calcium, which is the actual absorbable portion. Furthermore, as discussed herein, it has been suggested that magnesium is necessary for calcium uptake and energy production, as well as for the correct function of osteoblasts (i.e., bone building cells). Studies show that 80% of Americas do not get the required daily amount of magnesium. Studies also indicate a 2:1 balance of calcium to magnesium is optimal and is generally the normal ratio of calcium to magnesium found in the human body. Thus, a supplement dosage of 1000 mg of calcium may only yield a small absolute amount of absorbable calcium. Moreover, if the calcium is combined with magnesium, the simultaneous competitive absorption of the magnesium component will further decrease the amount of calcium that is actually absorbed.

Magnesium is the second most abundant intracellular cation in vertebrates. About half the total magnesium in the body of a normal adult human is present intracellularly in soft tissues, and the other half is in bone. Interactions between calcium and magnesium absorption in the intestine have been reported. Based on in vitro experiments, some researchers have suggested that magnesium and calcium compete with each other for intestinal absorption.

Research that examined bone loss in postmenopausal women reported that magnesium supplementation significantly increased bone density or arrested bone loss in postmenopausal osteoporosis. Such research also reported that a significant increase in density of the calcaneus bone was observed when postmenopausal women on hormonal replacement therapy were supplemented with magnesium.

While such research has shown that magnesium supplementation reduces apparent calcium absorption, it also promoted bone formation and prevents bone resorption. That is, it individually affects intestinal calcium absorption and bone metabolism. Moreover, such research has shown that magnesium supplementation increases the dynamic strength of bone.

From infancy to old age, calcium is emphasized as an important building block and is the most abundant mineral in the human body. As previously discussed, calcium provides strength to bones and teeth, in which nearly 99% of the body's calcium is stored. The remaining calcium, which is in the bloodstream, assists in muscle contraction, blood clotting and nerve impulse management. It also assists in the regulation of heartbeat, and assists with short-term memory. If calcium is not provided to the body by means of natural foods or supplements, the body will “steal” calcium from the bones to ensure that the required amount of calcium is present in the bloodstream. If the body is not given enough calcium from natural foods or supplements and is forced to take it from the bones and teeth, a “negative calcium balance” will result.

Sources of calcium loss in the body include urine, sweat, feces etc. When the body becomes deficient of calcium, a key result is bone mass loss. In post-menopausal women particularly, calcium absorption drops off after menopause, which results in bone mass loss. Over thirty (30) million Americans now suffer from low bone mass disease, which can lead to fractures, complications and eventual long-term hospitalization.

In monetary terms, calcium has immense benefits to public health in general. For example, maintaining proper calcium levels prevents tooth and bone decay, the treatment of which costs the United States over $10 billion per year in osteoporosis related research and treatment. Recent studies by a Harvard University team indicated that raising calcium levels to the recommended daily amount could also lower the risk of some types of colon cancer by 50%, thus expanding the impact of calcium to many areas of human life.

As discussed above, studies show that half of the American population is estimated to consume less than 600 mg of calcium daily. Americans typically get about 75% of their dietary calcium from dairy products such as milk, yogurt, cheese, etc. However, recent research from the Physicians Health Study at Harvard University indicates that the most common dietary calcium source (dairy products) is linked to increased risk of prostate cancer. For women, hormone replacement that once helped women absorb calcium after menopause is now being discontinued because of side effects. This further creates a new need for what research shows is a 500 mg per/day calcium gap due to the discontinuation of hormone replacement.

As discussed above, the recommended daily allowance of calcium for adults is approximately 1000 mg elemental calcium. Adult subjects however usually get only on the order of 500-600 mg of calcium per day from natural food sources, such as dairy products and the like. For an average “American” diet, it is quite difficult to get all of the daily required calcium from a diet of natural foods. Thus, a calcium supplement is required to help a subject obtain the required 1000 mg or more of calcium per day. The effectiveness of a calcium supplement is, however, highly affected by the simultaneous intake of other minerals, such as magnesium.

If calcium is taken in supplement form, as previously emphasized, the dosage must be in the right form and quantity, and must be tailored to avoid side effects. Supplemental calcium must also be in the right combination of vitamins and minerals to support absorption and use by the body.

With regard to the form of calcium, calcium carbonate and calcium phosphate are not as attractive as calcium citrate. Calcium carbonate must be taken with food to help reduce side effects (i.e. upset stomach or gas) and encourage absorption (i.e. food causes HCL acid to be produced, which enhances absorption for calcium carbonate). Some reports have indicated the presence of lead in some calcium carbonate supplements, although the quantity is below legal thresholds. However, the use of calcium carbonate is the cheapest approach and therefore most prevalent on the market. The use of calcium carbonate in antacid products is particularly questionable, given that the carbonate form needs acid to be absorbed, but a subject ends up taking antacid at the same time.

Calcium phosphate is sometimes touted as an option “closest to the body's natural bone” because bones contain calcium and phosphorus. However, in practice, the body has a harder time breaking down calcium phosphate in a pharmaceutical form.

Because calcium carbonate and calcium phosphate have a higher percentage of elemental calcium in them than calcium citrate (40% vs. 21%), many calcium supplements are marketed by boasting “the most calcium per pill.” In fact, all this claim means is that an individual has to consume more calcium citrate to get a same net 1000 mg of elemental calcium, not that the carbonate form is somehow better or stronger.

Due to the above-identified drawbacks of calcium carbonate and calcium phosphate, calcium citrate is considered to be a better form of calcium supplement for the following reasons. Calcium citrate has the highest bioavailability and does not need stomach acid to help absorption. Calcium citrate has been shown in some studies to be better absorbed than calcium carbonate and does not require food taken therewith. It also has a 27% higher absorption rate than calcium carbonate when both are taken on an empty stomach and 22% higher absorption rate versus calcium carbonate when both are taken with food. Calcium citrate is also the only form of calcium that can be absorbed without stomach acid.

The problem, however, in taking a typical magnesium/calcium supplement, however, is that the calcium and magnesium compete for absorption. Thus, competitive absorption is a problem that exists following the oral ingestion of a mixture containing both the minerals calcium and magnesium. Ideally these two minerals should be ingested separately, and at different times, in order to maximize the minerals' absorption in the small intestine.

In a typical supplement formulation containing both magnesium and calcium, upon oral ingestion, both minerals are released from the mixture for absorption at relatively the same time. Because calcium and magnesium utilize the same absorption process in the small intestine, there is competition between the two minerals for absorption. Due to this competition, neither mineral is optimally absorbed. Thus, even though a supplement may contain a quantity of both calcium and magnesium in an amount that if individually administered may provide a sufficient dose so as to be effective, the resulting competition of the two minerals during the digestion and absorption process results in a less than recommended dose being absorbed into the body.

Thus, it would be advantageous to provide a nutritional supplement for oral ingestion that contains both calcium and magnesium that delays absorption of either the calcium or magnesium in order to allow absorption of one of the minerals before absorption of the other.

### SUMMARY OF THE INVENTION

Accordingly, the present invention provides a nutritional supplement comprised of calcium and magnesium combined in a nutritional mixture for oral ingestion. In one embodiment, the release of magnesium for absorption in the body is delayed for a period of time (e.g., six to eight hours) after ingestion of the nutritional supplement. This may be accomplished by using a magnesium salt, such as magnesium chloride, that has been microencapsulated to provide a targeted delayed time of release relative to the calcium. Such microencapsulation may be accomplished using various coating materials known in the art such as ethyl cellulose combined with a plasticizer. The amount and type of coating material and plasticizer can be selected to reach the delayed release time of six to eight hours following ingestion thereof. The microencapsulated magnesium can be combined with a calcium salt such as calcium citrate.

The delay of absorption of the magnesium alleviates the problem associated with the competitive absorption that exists between calcium and magnesium when the two are ingested together in a nutritional mixture.

In one embodiment of a nutritional supplement according to the principles of the present invention, a magnesium/calcium supplement is prepared in a manner in which the calcium is microencapsulated and the magnesium is not so that the magnesium is absorbed first in the small intestines of the subject. Through microencapsulation, the calcium is delayed for a period of time (e.g., six to eight hours) after ingestion of the nutritional supplement.

In another embodiment, the nutritional supplement according to the principles of the present invention is formed in a manner in which the magnesium is encapsulated relative to the calcium such that the calcium can be substantially fully absorbed prior to the release of the magnesium. This may be accomplished by using liquid filled capsules in which the magnesium is separately encapsulated within an outer encapsulated calcium mixture. As such, the calcium can be released in a first stage of release in which the outer shell of the capsule is dissolved first within the digestive track of the user and a shell of an inner capsule is dissolved at a later time to release the magnesium. The delay between the release of the calcium and the magnesium can be controlled by adjusting the properties of the inner capsule to provide release of the magnesium after a relatively controlled period of time.

A nutritional supplement in accordance with the present invention may be provided in doses that are generally recommended. That is, for an average adult in generally good health, the recommended daily dosage is approximately 1000 mg calcium and approximately 500 mg magnesium. For older adults with osteoporosis, for post-menopausal women or for individuals with a calcium deficiency, the recommended daily dosage is approximately 1200 mg to 2000 mg calcium and approximately 1000 mg magnesium. Because of the increased absorption of both calcium and magnesium as a result of the time delay characteristics of the nutritional supplement of the present invention, the amount of calcium and/or magnesium need not be increased to accommodate for the competitive absorption that would otherwise occur if the two minerals were trying to be absorbed simultaneously. Thus, one can be assured with the nutritional supplement of the present invention that the person taking the supplement is receiving the recommended dose of each mineral since the supplement of the present invention eliminates, or at least substantially decreases the amount of competitive absorption in the small intestines.

The nutritional supplement of the present invention can be used to prepare typical oral dosage forms such as filled hard gelatin capsules, soft gelatin capsules, compressed or otherwise formed tablets. The nutritional supplement may also be combined with various other ingredients such as other vitamins or minerals and fillers, binders and coatings known in the art.

The calcium/magnesium nutritional supplement of the present invention may also be incorporated into various other forms for oral administration. For example, the nutritional supplement may be included with various fillers and flavorings in the form of a powdered drink mix, a chewable tablet or soft chew, or other forms of oral ingestion known in the art.

Additional features, advantages, and embodiments of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 is a schematic block diagram of a first embodiment of a method of providing a nutritional supplement containing calcium and magnesium in accordance with the principles of the present invention;

FIG. 2 is a schematic block diagram of a second embodiment of a method of providing a nutritional supplement containing calcium and magnesium in accordance with the principles of the present invention;

FIG. 3 is a cross-sectional side view of a first embodiment of a nutritional supplement containing calcium and magnesium in accordance with the principles of the present invention; and

FIG. 4 is a cross-sectional side view of a second embodiment of a nutritional supplement containing calcium and magnesium in accordance with the principles of the present invention.

### DETAILED DESCRIPTION

The present invention provides a composition and method for dietary mineral supplementation utilizing a form and dosage of calcium and magnesium for enhanced calcium and magnesium absorption.

More particularly, in one embodiment, the present invention provides a method and composition for delaying the absorption of the magnesium for a period of time after absorption of the calcium component of the supplement. The supplement provided herein facilitates absorption of calcium in bones of a subject for enhanced physiological and psychological benefits. The supplement is especially beneficial for subjects experiencing osteoporosis, arthritis, demineralization of bones, bodily pain and lack of energy, as well as for the prevention of these ailments.

Because of competitive absorption in the small intestine that would otherwise occur when magnesium and calcium are taken simultaneously, the present invention provides a nutritional supplement comprised of calcium and magnesium combined in a nutritional supplement that delays the absorption time of one of the magnesium or the calcium for a period of time to allow the first to be absorbed mineral to be sufficiently absorbed in the small intestine before absorption of the other.

The delay of absorption of one of the magnesium or calcium relative to the other alleviates the problem associated with the competitive absorption that exists between calcium and magnesium when the two are ingested together in a nutritional mixture.

As is discussed with reference to the drawings, the release of magnesium for absorption in the body is delayed for a period of time (e.g., six to eight hours) after ingestion of a nutritional supplement also containing a form of calcium, such as calcium citrate. This may be accomplished by using a magnesium salt, such as magnesium chloride, that has been microencapsulated or encapsulated to provide a targeted delayed time of release relative to the calcium. Such microencapsulation or encapsulation may be accomplished using various coating materials for minerals known in the art such as ethyl cellulose combined with a plasticizer. The amount and type of coating material and plasticizer can be selected to reach the delayed release time of six to eight hours following ingestion thereof.

In another exemplary embodiment, a magnesium/calcium supplement is prepared in a manner in which the calcium is delayed for absorption relative to the magnesium so that the magnesium is absorbed first in the small intestines of the subject. Through microencapsulation or encapsulation, the calcium is delayed for a period of time (e.g., six to eight hours) after ingestion of the nutritional supplement.

The optimal or desired delay of absorption of one or more ingredients of the present invention may be accomplished by using liquid filled capsules in which the magnesium or calcium is separately encapsulated relative to an outer encapsulated calcium or magnesium mixture. As such, the calcium or magnesium, as the case may be, can be released in a first stage of release in which the outer shell of the capsule is dissolved first within the digestive track of the subject and an inner capsule is dissolved at a later time to release the other mineral. The delay between the release of the calcium and the magnesium can be controlled by adjusting the properties of the encapsulation to provide release after a relatively controlled period of time.

The nutritional supplement is provided in doses that are generally recommended for adults in various states of health. For example, the recommended daily dose for a healthy adult is approximately 1000 mg calcium and approximately 500 mg magnesium. For older adults with osteoporosis or for post-menopausal women, the recommended daily dose is approximately 1200 mg to 2000 mg calcium and approximately 1000 mg magnesium. Because of the increased absorption of both calcium and magnesium as a result of the time delay characteristics of the nutritional supplement of the present invention, however, the amount of calcium and/or magnesium need not be increased to accommodate for the competitive absorption that would otherwise occur if the two minerals were being absorbed simultaneously. Indeed, the lack of competitive absorption of the two minerals may result in lower required doses that would otherwise be required. Thus, one can be assured with the nutritional supplement of the present invention that the person taking the supplement is receiving at a minimum the recommended dose of each mineral since the supplement of the present invention eliminates, or at least substantially decreases the amount of competitive absorption in the small intestines of the two minerals.

The nutritional supplement of the present invention can be provided in various oral dosage forms such as filled hard gelatin capsules, soft gelatin capsules, or compressed or otherwise formed tablets. The nutritional supplement may also be combined with various other ingredients such as other vitamins or minerals and fillers, binders and coatings known in the art.

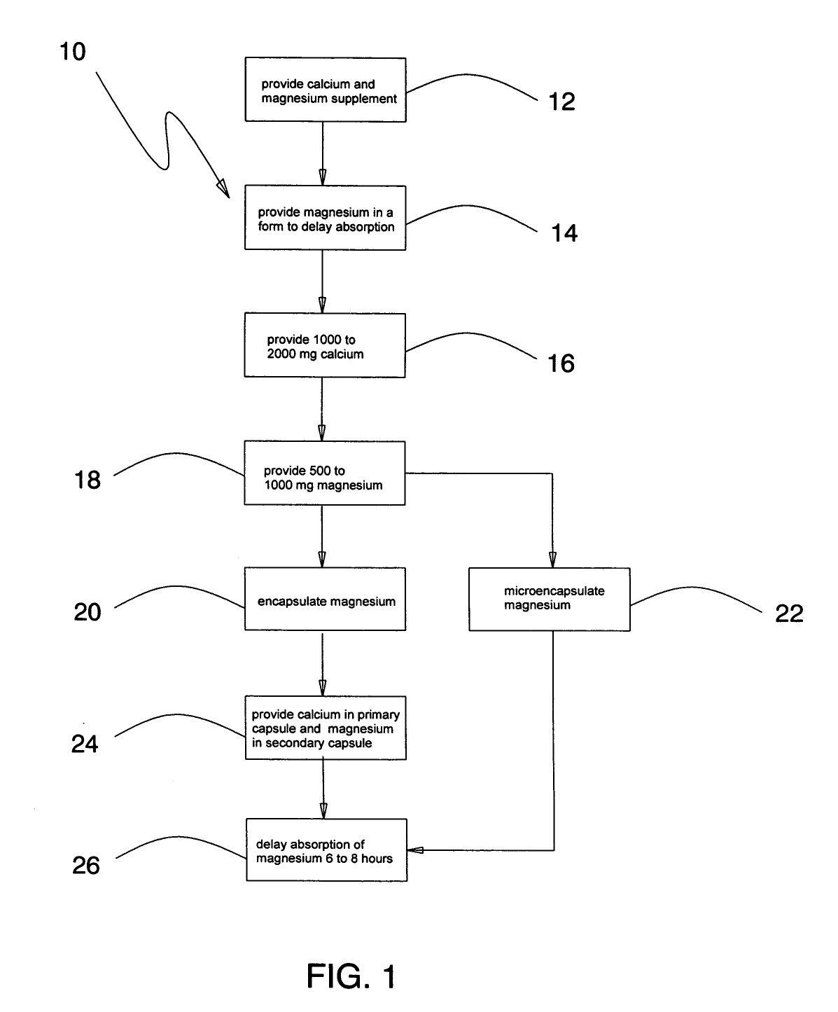

Referring now to FIG. 1, there is illustrated a method, generally indicated at 10, of providing a nutritional supplement 12 containing both calcium and magnesium. The magnesium is provided 14 in a form to delay absorption of the magnesium for a period of time after ingestion of the nutritional supplement. The calcium contained within the nutritional supplement is provided 16 in a therapeutically effective dose depending generally upon the health and condition of the particular person. Generally, the dosage ranges from about 1000 mg to 2000 mg. For a normal healthy adult, a dosage of approximately 1000 mg per day of calcium is recommended to prevent osteoporosis and other ailments resulting from a calcium deficiency. For persons with osteoporosis, post-menopausal women or individuals suffering from other calcium deficiencies, such as a decrease in bone density and/or dynamic bone strength, a daily dosage of between about 1200 mg and 2000 mg or higher will likely be prescribed.

The magnesium is provided 18 in therapeutically effective doses generally depending upon the health and condition of the subject taking the nutritional supplement. A daily dosage of approximately between 500 mg and 1000 mg is generally recommended for most adults depending upon their particular state of health. For a normal healthy adult, a daily dose of approximately 500 mg of magnesium is in most cases sufficient to maintain health and to prevent ailments that may arise as a result of a magnesium deficiency. For persons suffering from an ailment that is a result of a magnesium deficiency, a daily dose of approximately 1000 mg or higher of magnesium will likely be prescribed.

In order to eliminate or at least significantly decrease competitive absorption of the calcium and magnesium contained in the nutritional supplement of the present invention, the magnesium may be encapsulated 20 or microencapsulated 22 to allow the calcium to be absorbed first in the small intestines of the subject taking the supplement. By encapsulating or microencapsulating the magnesium, digestion of the encapsulating or microencapsulating coating of the magnesium will occur at some period of time after digestion and absorption of the calcium. When present in the small intestines of the subject taking the supplement, the magnesium can be available for absorption after the calcium as been sufficiently absorbed.

One way of encapsulating 20 the magnesium is to provide 24 the calcium in a primary capsule and the magnesium in a secondary capsule. The primary capsule is configured to quickly dissolve in the stomach of the person taking the supplement upon oral ingestion. In that way, the calcium becomes quickly available for absorption in the small intestines. The secondary capsule is configured to provide a time release of the magnesium after a period of time has elapsed after ingestion of the supplement by the subject. When the shell or coating defining the secondary capsule has adequately dissolved, the magnesium will be available for absorption. This time delay allows the subject's digestive track to absorb the calcium for a period of time prior to the magnesium being available for similar absorption. The delay 26 of absorption of the magnesium is between approximately six to eight hours after ingestion of the nutritional supplement to allow adequate time for absorption of the calcium component of the supplement. It is also contemplated, in accordance with the present invention, that the time of delay may be adjusted to be shorter by simply modifying the properties of the material encapsulating 20 or microencapsulating 22 the magnesium.

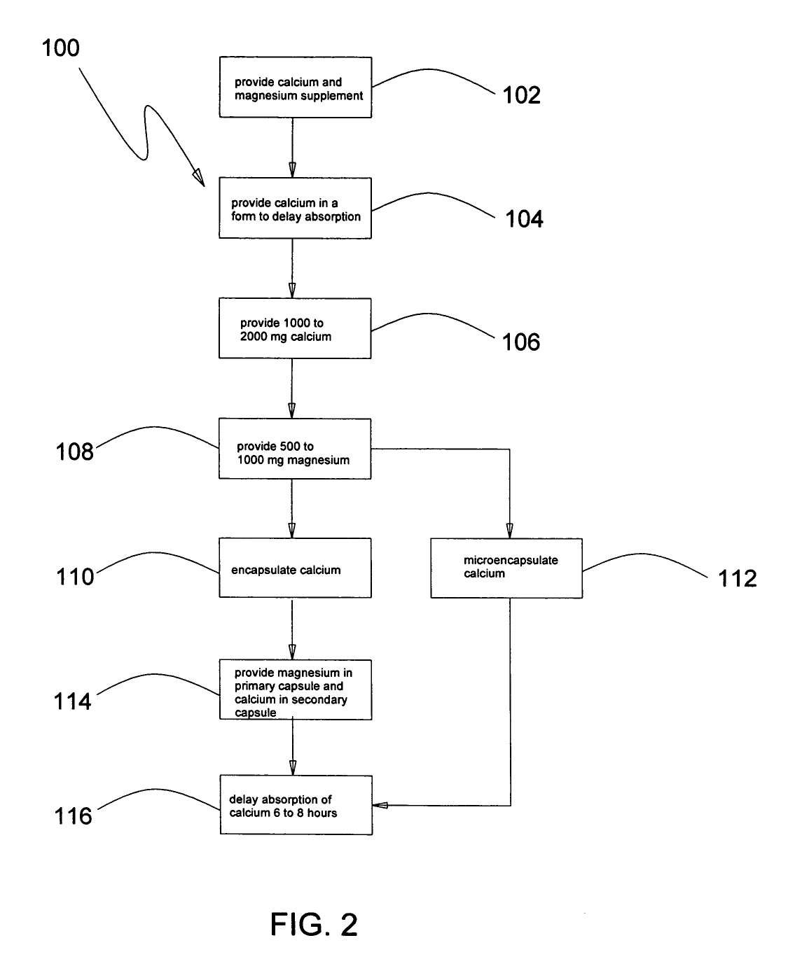

Because the calcium and magnesium compete for the same absorption sites within the small intestine, it is important, in accordance with the principles of the present invention, to delay the absorption of one of the magnesium or calcium relative to the other. FIG. 2 illustrates a similar method to that illustrated with reference to FIG. 1, but where the calcium component of a supplement in accordance with the principles of the present invention is delayed for absorption after the nutritional supplement has been ingestion and the magnesium component has been sufficiently absorbed.

As shown in FIG. 2, a method, generally indicated at 100, of providing a nutritional supplement 102 containing both calcium and magnesium. The calcium is provided 104 in a form to delay absorption of the calcium for a period of time after ingestion of the nutritional supplement. The calcium contained within the nutritional supplement is provided 106 in a therapeutically effective amount of between about 1000 mg and 2000 mg. For a normal healthy adult, a dosage of approximately 1000 mg per day of calcium is recommended to prevent osteoporosis and other ailments resulting from a calcium deficiency. For persons with osteoporosis, post-menopausal women or individuals suffering from other calcium deficiencies, such as a decrease in bone density and/or dynamic bone strength, a daily dosage of between about 1200 mg and 2000 mg or higher will likely be prescribed.

The magnesium is provided 108 in a therapeutically effective daily dosage of approximately between 500 mg and 1000 mg. For a normal healthy adult, a daily dose of approximately 500 mg of magnesium is in most cases sufficient to maintain heath and to prevent ailments that may arise as a result of a deficiency in magnesium. For persons suffering from an ailment that is a result of a magnesium deficiency, a daily dosage of approximately 1000 mg or higher of magnesium will likely be prescribed.

In order to eliminate or at least significantly decrease competitive absorption of the calcium and magnesium contained in the supplement, the calcium may be encapsulated 110 or microencapsulated 112 to allow the calcium to be absorbed first in the intestines of the subject taking the supplement. By encapsulating or microencapsulating the calcium, digestion of the encapsulating or microencapsulating coating of the calcium will occur at some period of time after digestion and absorption of the magnesium. When present in the small intestines of the subject taking the supplement, the calcium can be available for absorption after the magnesium has been sufficiently absorbed.

One way of encapsulating 110 the calcium is to provide 114 the magnesium in a primary capsule and the calcium in a secondary capsule. The primary capsule is configured to quickly dissolve in the stomach of the subject taking the supplement upon oral ingestion. In that way, the magnesium becomes quickly available for absorption in the small intestines. The secondary capsule is configured to provide a time release of the calcium after a period of time has elapsed after ingestion of the supplement by the individual. When the shell or coating defining the secondary capsule has adequately dissolved, the calcium will be available for absorption. This time delay allows the subject's digestive track to absorb the magnesium for a period of time prior to the calcium being available for similar absorption. The delay 116 of absorption of the calcium is between approximately six to eight hours after ingestion of the nutritional supplement to allow adequate time for absorption of the magnesium component of the supplement. It is also contemplated, in accordance with the present invention, that the time of delay may be adjusted to be shorter by simply modifying the properties of the material encapsulating 110 or microencapsulating 112 the calcium.

The calcium and magnesium contained in a nutritional supplement for oral ingestion in accordance with the principles of the present invention are of the type known in the art as being generally ingestible and absorbable through the digestive track of a person. Such forms of calcium may comprise calcium salt such as calcium citrate, calcium carbonate, calcium phosphate or other forms of calcium for oral ingestion known in the art. The magnesium may comprise magnesium salt, such as magnesium chloride, magnesium phosphate dibasic or other forms of magnesium for oral ingestion known in the art.

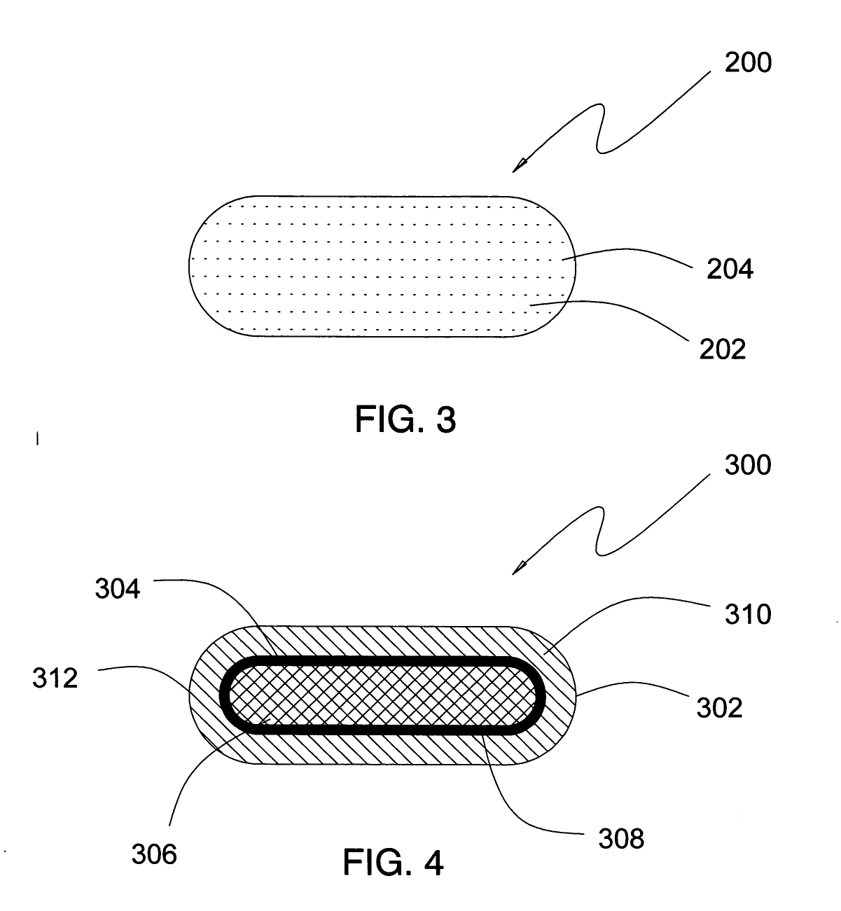

As shown in FIG. 3, a nutritional supplement, generally indicated at 200 in accordance with the principles of the present invention, may comprise calcium 202 and microencapsulated magnesium 204. As previously discussed, the magnesium may be microencapsulated with a coating configured for delayed time release within the digestive track of the person taking the supplement 200, such as an ethyl cellulose material combined with a plasticizer. The amount and type of coating material and plasticizer can be selected to reach the desired delayed release time, for example, six to eight hours after ingestion of the nutritional supplement 200.

It should also be noted that the nutritional supplement 200 is not limited to containing only magnesium and calcium. Indeed, the nutritional supplement may contain other ingredients including other minerals or vitamins such as vitamin D, vitamin C, and others. The nutritional supplement can be prepared in typical oral dosage forms such as filled hard gelatin capsules, soft gelatin capsules, tables, such as compressed tablets, or other forms for oral ingestion known in the art.

Referring now to FIG. 4, there is illustrated a capsule, generally indicated at 300, in accordance with the present invention. The capsule 300 is comprised of a primary capsule 302 and a secondary capsule 304. Depending upon the ingredient for which a time delay is desired, the first to be absorbed ingredient is provided between the outside of the secondary capsule 304 and the inside of the primary capsule 302. If a calcium component is to be absorbed first, magnesium 306 is provided in the secondary capsule 302. The secondary capsule is defined by an outer wall or shell 308 having properties for delaying digestion of the magnesium 306 for a period of time after the calcium component 310 contained in the primary capsule has been dissolved and absorbed. Thus, the shell 312 of the primary capsule is of a thickness and material that allows for rapid digestion of the calcium 310 after ingestion of the capsule 300. Once the calcium 310 has been dissolved from the capsule, the shell 304 of the secondary capsule can begin to be digested resulting in a time delay between the availability for absorption of the calcium 310 and magnesium 306. Of course, if the calcium 310 is to be delayed after ingestion of the capsule 310 relative to the absorption of the magnesium component, the calcium would be provided within the inner secondary capsule 304 and the magnesium would be contained in the outer primary capsule 302.

It is further contemplated that the calcium/magnesium nutritional supplement of the present invention may be incorporated into various forms for oral ingestion. For example, the nutritional supplement may comprise a powdered drink mix which includes various fillers and flavorings known in the art. The powdered drink mix would provide a delayed absorption of either the calcium or magnesium in accordance with the principles of the present invention discussed previously herein. For example, the magnesium or calcium may be included in the powdered drink mix in a microencapsulated form so as to delay the absorption of the microencapsulated mineral. Once the drink mix was prepared into a liquid form as by the addition of water, the drink mix should be ingested within a relatively short period of time, since the timed release of the microencapsulated mineral could begin upon the addition of water to the powdered drink mix, not the time of ingestion. Thus, the powdered drink mix could include instructions indicating that the drink mix should be ingested upon preparation of the drink mix in liquid form.

It is further contemplated that the nutritional supplement of the present invention may be prepared in various other forms for oral ingestion, including without limitation chewable tablets or soft chews, mixable liquids, combination liquid and solid forms of the minerals for mixing and ingestion or other forms of oral ingestion known in the art. The claims appended hereto are intended to cover all such forms of a nutritional supplement in accordance with the principles of the present invention.

Those of skill in the art will appreciate upon a review and understanding of the present invention that there may be other methods and techniques for delaying the absorption of one ingredient of a nutritional supplement relative to another in order to allow a first ingredient to be sufficiently absorbed prior to a second ingredient being available for absorption. The present invention and the claims herein are intended to cover such other methods and techniques. Thus, although particular embodiments and examples of the invention have been described in detail herein, it is to be understood that the invention is not limited to those particular embodiments and examples, and that various changes and modifications may be affected therein by one skilled in the art without departing from the scope or spirit of the invention as defined in the appended claims.

What is claimed is:

1. A method for dietary calcium and magnesium supplementation, comprising:
providing a pharmaceutically acceptable composition comprised of calcium in an ingestible form and magnesium in an ingestible form; and
providing the ingestible form of magnesium in a form that will delay absorption of the magnesium after at least some absorption of the ingestible form of calcium.

2. The method of claim 1, further comprising microencapsulating said magnesium to provide the delayed absorption of the magnesium relative to the calcium.

3. The method of claim 1, further comprising encapsulating said magnesium to provide the delayed absorption of the magnesium relative to the calcium.

4. The method of claim 3, further comprising providing said calcium in a primary capsule and said magnesium in a secondary capsule, said secondary capsule having a longer dissolving time than said primary capsule so as to allow absorption of said calcium prior to absorption of said magnesium.

5. The method of claim 1, further comprising providing a therapeutically effective daily dosage of said calcium of approximately 1000 mg and a therapeutically effective daily dosage of said magnesium of approximately 500 mg.

6. The method of claim 1, further comprising providing a therapeutically effective daily dosage of said calcium of approximately 1200 to 2000 mg and a therapeutically effective daily dosage of said magnesium of approximately 1000 mg.

7. The method of claim 1, further comprising delaying absorption of said magnesium approximately six to eight hours after ingestion of said calcium.

8. A nutritional supplement for enhancing the absorption of calcium and magnesium, comprising:
a therapeutically effective amount of calcium in an ingestible form; and
a therapeutically effective amount of magnesium in an ingestible form, said magnesium being encapsulated so as to delay the absorption of the magnesium relative to the calcium.

9. The nutritional supplement of claim 8, wherein said encapsulated magnesium comprises microencapsulated magnesium.

10. The nutritional supplement of claim 8, further comprising a first capsule for containing said calcium and a second capsule for containing said magnesium.

11. The nutritional supplement of claim 10, wherein said first and second capsules are dissolved at different times within an ingestion track of a person so as to delay absorption of said magnesium relative to said calcium.

12. The nutritional supplement of claim 8, wherein said calcium comprises a therapeutically effective daily dosage of calcium of approximately 1000 mg and wherein said magnesium comprises a therapeutically effective daily dosage of magnesium of approximately 500 mg.

13. The nutritional supplement of claim 8, wherein said calcium comprises a therapeutically effective daily dosage of calcium of approximately 1200 to 2000 mg and said magnesium comprises a therapeutically effective daily dosage of approximately 1000 mg.

14. The nutritional supplement of claim 8, wherein said encapsulated magnesium is configured to provide delayed absorption of said magnesium approximately six to eight hours after ingestion of said calcium.

15. A method for dietary calcium and magnesium supplementation, comprising:
providing a pharmaceutically acceptable composition comprised of calcium in an ingestible form and magnesium in an ingestible form; and
providing the ingestible form of calcium in a form that will delay absorption of the calcium after at least some absorption of the ingestible form of magnesium.

16. The method of claim 15, further comprising microencapsulating said calcium to provide the delayed absorption of the calcium relative to the magnesium.

17. The method of claim 15, further comprising encapsulating said calcium to provide the delayed absorption of the calcium relative to the magnesium.

18. The method of claim 17, further comprising providing said magnesium in a primary capsule and said calcium in a secondary capsule, said secondary capsule having a longer dissolving time than said primary capsule so as to allow absorption of said magnesium prior to absorption of said calcium.

19. The method of claim 15, further comprising providing a therapeutically effective daily dosage of said calcium of approximately 1000 mg and a therapeutically effective daily dosage of said magnesium of approximately 500 mg.

20. The method of claim 15, further comprising providing a therapeutically effective daily dosage of said calcium of approximately 1200 to 2000 mg and a therapeutically effective daily dosage of said magnesium of approximately 1000 mg.

21. The method of claim 15, further comprising delaying absorption of said calcium approximately six to eight hours after ingestion of said magnesium.

22. A nutritional supplement for enhancing the absorption of calcium and magnesium, comprising:
a therapeutically effective amount of magnesium in an ingestible form; and
a therapeutically effective amount of calcium in an ingestible form, said calcium being encapsulated so as to delay the absorption of the calcium relative to the magnesium.

23. The nutritional supplement of claim 22, wherein said encapsulated calcium comprises microencapsulated calcium.

24. The nutritional supplement of claim 22, further comprising a first capsule for containing said magnesium and a second capsule for containing said calcium.

25. The nutritional supplement of claim 24, wherein said first and second capsules are dissolved at different times within an ingestion track of a person so as to delay absorption of said calcium relative to said magnesium.

26. The nutritional supplement of claim 22, wherein said calcium comprises a therapeutically effective daily dosage of calcium of approximately 1000 mg and wherein said magnesium comprises a therapeutically effective daily dosage of magnesium of approximately 500 mg.

27. The nutritional supplement of claim 22, wherein said calcium comprises a therapeutically effective daily dosage of calcium of approximately 1200 to 2000 mg and said magnesium comprises a therapeutically effective daily dosage of approximately 1000 mg.

28. The nutritional supplement of claim 22, wherein said encapsulated calcium is configured to provide delayed absorption of said calcium approximately six to eight hours after ingestion of said magnesium.
